(19) United States
(12) Patent Application Publication
(10) Pub. No.: US 2004/0242471 A1
(43) Pub. Date: Dec. 2, 2004

(54) Dormancy induced mycobacterium proteins

(76) Inventors: Edwin Esser, Dietzenbach (DE); Hans Wedel, Muhltal (DE)

Correspondence Address: COHEN, PONTANI, LIEBERMAN & PAVANE, 551 FIFTH AVENUE, SUITE 1210, NEW YORK, NY, 10176, US

(21) Appl. No.: 10/450,726

(22) PCT Filed: Dec. 11, 2001
(86) PCT No.: PCT/EP01/14551

(30) Foreign Application Priority Data
Dec. 13, 2000 (GB) 0030368.5

(51) Int. Cl. G01N 33/554; G01N 33/569; A61K 38/16

(52) U.S. Cl. 514/12; 435/7.32

(57) ABSTRACT

A method for the identification of an anti-mycobacterial agent that modulates the activity and/or expression of a protein expressed by a Mycobacterium in non-oxygen limiting stationary phase, hypoxic stationary phase or hypoxic growth phase, which method comprises: (i) contacting a test agent and a protein selected from RV3133c, Rv2623, Rv2626c, a variant of Rv3133c, Rv2623 or Rv2626 and a fragment of Rv3133c, RV2623, Rv2626c or said variant, or a polynucleotide or expression vector encoding said protein; (ii) monitoring the effect of the test agent on the activity and/or expression of said protein, thereby determining whether the test agent modulates the activity and/or expression of a protein expressed by a Mycobacterium in non-oxygen limiting stationary phase, hypoxic stationary phase or hypoxic growth phase.

### FIELD OF THE INVENTION

The present invention relates to proteins expressed in dormant Mycobacterium and to the use of such polypeptides in the prophylaxis, diagnosis and treatment of mycobacterial infections.

### BACKGROUND TO THE INVENTION

Upon infection with Mycobacterium tuberculosis most individuals mount an effective immune response causing the infection to enter a latent state. The tubercle bacillus may remain in the host for years without causing the symptoms of tuberculosis, with possible reactivation later in life. Little is known about the state in which the bacilli survive in the host during latent infection. Some evidence suggests that cells survive in a state that is similar to the state of bacilli in non-oxygen limiting or hypoxic stationary phase culture.

Mycobacteria are obligate aerobes, i.e. they require oxygen for growth. However, tubercle bacilli encounter hypoxic environments in acute disease as well as in latent infection. Recent genetic evidence suggests that the capability of tubercle bacilli to adapt to hypoxic conditions plays a role in vivo. A link has been established between oxygen starvation and drug resistance. Upon depletion of oxygen in culture the bacillus enters a hypoxic growth phase then terminates growth and develops into a dormant form. Importantly, the dormant form of the bacterium is resistant against conventional anti-mycobacterials. Hence, hypoxic dormant bacteria could, at least in part, be responsible for the observed persistence of the pathogen during chemotherapy.

The Wayne dormancy culture system (Wayne and Hayes (1996) Infect. Immun. 64: 2062-2069) is based on growth of the bacilli under oxygen-limited conditions in sealed stirred tubes. Initially the cultures grow exponentially and consume oxygen rapidly. A temporal oxygen gradient is generated and the cultures terminate growth when the oxygen concentration reaches a hypoxic threshold level. Bacilli in the hypoxic stationary phase are in a state of synchronised non-replicating persistence or dormancy.

Our knowledge of the molecules involved in the mycobacterial dormancy response is fragmentary. Elegant biochemical work has shown that dormant bacilli adapt their metabolism to anaerobiosis by switching to nitrate respiration and reductive amination of glyoxylate. Genetic analysis has demonstrated that the stringent response plays a crucial role in the adaptation to hypoxic conditions and stationary phase survival.

### SUMMARY OF THE INVENTION

The present inventors have identified four proteins that are up-regulated upon termination of growth of the attenuated BCG Pasteur ATCC 35734 strain of Mycobacterium bovis in the Wayne dormancy culture system. The inventors have also shown that two of these proteins are upregulated in Mycobacterium bovis maintained in a non-oxygen limiting stationary phase. These proteins play a role in the development of the dormant state and in the maintenance of viability during dormancy. The proteins are novel screening targets for the identification and development of novel pharmaceutical agents. These agents may be used in the treatment, prophylaxis and/or diagnosis of mycobacterial infections such as tuberculosis.

Accordingly, the present invention provides:

a method for the identification of an anti-mycobacterial agent that modulates the activity and/or expression of a protein, which method comprises:

(i) contacting a test agent and a protein selected from RV3133c, Rv2623, Rv2626c, a variant of Rv3133c, Rv2623 or Rv2626c and a fragment of Rv3133c, RV2623, Rv2626c or said variant, or a polynucleotide or expression vector encoding said protein;

(ii) monitoring the effect of the test agent on the activity and/or expression of said protein, thereby determining whether the test agent modulates the activity and/or expression of a protein expressed by a Mycobacterium in non-oxygen limiting stationary phase, hypoxic stationary phase or hypoxic growth phase.

an agent which is identifiable by a method of the invention;

an antibody specific for a protein selected from Rv3133c, Rv2623 or Rv2626c;

a pharmaceutical composition comprising a pharmaceutically effective carrier and as an active ingredient an effective amount of an agent or an antibody according to the invention;

a vaccine composition comprising as an active ingredient an effective amount of a protein selected from Rv3133c, Rv2623, Rv2626c and a variant of any thereof, or an immunogenic fragment any said protein, and a pharmaceutically effective carrier;

an agent, antibody, pharmaceutical composition or vaccine composition according to the invention for use in a method of treatment of the human or animal body by therapy or in a diagnostic method practised on the human or animal body;

use of an agent, antibody, pharmaceutical composition or vaccine composition according to the invention in the manufacture of a medicament for the diagnosis, prophylaxis or treatment of a mycobacterial infection;

a method of treating a subject suffering from a mycobacterial infection, which method comprises administering to said subject a therapeutically effective amount of an agent, antibody, pharmaceutical composition or vaccine composition according to the invention;

a method for detecting a mycobacterial infection in a sample, which method comprises detecting the presence of a protein expressed by a Mycobacterium in non-oxygen limiting stationary phase, hypoxic stationary phase or hypoxic growth phase or a nucleic acid encoding said protein in said sample, wherein said protein is selected from Rv3133c, Rv2623, RV2626c and a variant of any thereof, and

an in vitro or in vivo method for diagnosing a mycobacterial infection in a subject which method comprises monitoring expression of a protein expressed by a Mycobacterium in non-oxygen limiting stationary phase, hypoxic stationary phase or hypoxic growth phase, wherein said protein is selected from Rv3133c, Rv2623, RV2626c and a variant of any thereof.

use of a protein selected from Rv3133c, Rv2623, Rv2626c, a variant of Rv3133c, Rv2623 or Rv2626c and a fragment of Rv3133c, Rv2623, Rv2626c or said variant in a method for the identification of an anti-mycobacterial agent.

use of a protein selected from Rv3133c, Rv2623, Rv2626c, a variant of Rv3133c, Rv2623 or Rv2626c and a fragment of Rv3133c, Rv2623, Rv2626c or said variant in a method for the identification of an agent for diagnosing a dormant mycobacterial infection.

Preferably the mycobacterial infection is tuberculosis.

### BRIEF DESCRIPTION OF THE FIGURES

FIG. 1. shows the growth of BCG under the conditions of the Wayne dormancy culture system. Log A₆₀₀ as a function of time is shown. Viable counts at selected time points are indicated. An aerobic exponential preculture was diluted to A₆₀₀=0.005 with Dubos Tween-albumin broth (Difco) and incubated in sealed tubes under gentle stirring conditions. f and d indicate fading and complete decolorization of the oxygen indicator methylene blue. Mean values and standard deviations from four independent experiments are shown. Viable counts were determined by plating appropriate dilutions of the cultures on Dubos oleic albumin agar. For all cfu determinations, cultures were checked microscopically for any clumping of cells. Significant clumping was not observed. Arrows A to D indicate time points when samples were talcen for the two-dimensional electrophoretic analysis of protein contents shown in FIG. 2.

FIG. 2. shows the temporal profile of protein contents of BCG grown in the Wayne dormancy culture system. Protein extracts were prepared at the time points A to D indicated by the arrows in FIGS. 1 and 100 μg of total protein was subjected to two dimensional electrophoresis. A-D show silver-stained gels corresponding to the 4 time points. Arrows labeled 1 to 4 indicate dormancy-induced protein spots. The arrowhead shows the 22 kD alkyl hydroperoxide reductase (Ahpc, Rv2428) which has been found to be down-regulated in the hypoxic stationary phase (Sherman et al. (1999) Biofactors 10:211-217). Sizes are indicated in kilodaltons. The experiment was carried out four times with the same results. Growth phase-dependent protein contents were also analysed using isoelectric focussing strips pH 3-10. No additional dormancy-induced proteins were detected.

FIG. 3. shows dormancy-induced proteins. Protein numbers 1 to 4 correspond to protein spots 1 to 4 in FIG. 2. The protein names are according to the M. tuberculosis H37Rv genome annotation. To predict the domain architecture of the dormancy-induced proteins their sequence (derived from the M. tuberculosis H37Rv genome date base TubercuList (Institut Pasteur, Paris, France, [http://genolist.pasteur.fr/TubercuList/], Data Release R2, Mar. 22, 1999)) was searched against the protein domain families date base Pfam (Sanger Centre, UK[http://www.s.anger.ac.uk/Software/Pfam/], version 5.5 September 2000). The result of the search is shown schematically. HSP20, domain found in 20 kD family of heat shock proteins (Accession numberPF00011). res reg, receiver domain found in response regulators (Accession number PF00072). GerE, helix-turn-helix DNA binding domain (luxr subfamily) found in transcriptional regulators (Accession number PF00582). CBS, small modules of unknown function found in diverse proteins. Paire of CBS domains dimerise to form a stable globular domain (Accession number PF00571).

FIG. 4. shows steady state levels of mRNAs encoding dormancy-induced proteins in exponentially growing and dormant cultures. Autoradiograms of Northern blots of total RNA from exponentially growing (lane A, from time point A in FIG. 1) and hypoxic stationary phase cultures (lane D, from tine point D in FIG. 1) are shown. The blots were hybridised with [α⁻³²P]DATP labeled probes specific to the transcripts encoding the dormancy-induced proteins: 1, 16 kD antigen; 2, 23, kD response regulator; 3, 32 kD conserved hypothetical protein; 4, 16 kD conserved hypothetical protein (FIG. 3). X-ray films were exposed for 1 day. The bottom panel shows the blots after re-hybridisation with a probe specific to 16s rRNA demonstrating equal loading of ribosomal RNA. 5 μg of total RNA was loaded. The experiment was repeated once with the same results. The up-regulation of the transcripts was confirmed independently by reverse transcriptase —PCR analysis. Sizes are indicated in bases.

FIG. 5 shows the growth of BCG in roller bottles. Exponentially growing pre-cultures were diluted and grown in roller bottles. Optical density as a function of time is shown. Arrows A to D indicate time points when samples were taken for the two-dimensional gel electrophoretic analysis of protein contents showin in FIG. 6. The experiment was carried out four times with the same results and a representative growth curve is shown.

FIG. 6 shows the temporal profile of protein contents of BCG grown in roller bottles. Protein extracts were prepared at time points A to D indicated by the arrows in FIG. 5 and subjected to two dimensional gel electrophoresis. A-D show silver-stained gels corresponding to the 4 time points. Arrows labeled 1 and 2 indicate stationary phase-induced protein spots. The arrowhead shows the 22 kD alkyl hydroperoxide reductase (AhpC, Rv2428) which was found to be down-regulated in the stationary phase. The experiment was carried out four times and a representative set of gels is shown.

FIG. 7 shows a multiple sequence alignment of the 14 kD stationary phase and hypoxic stationary phase induced tubercle bacillus protein and the most similar orthologs found in other bacteria. The sequence of the 143 amino acid mycobacterial protein Rv2626c as derived from the M tuberculosis H37Rv genome data base TubercuList (Institut Pasteur, Paris, France, [http://genolist.pasteur.fr/TubercuList/]) was searched against the non-redundant GenBank data base (National Center for Biotechnology Information, Bethesda, USA, [http://www/ncbi.nlm.nih.gov]) to identify orthologs in other bacteria. M. tub.: 143 amino acid protein Rv2626c from Mycobacterium tuberculosis, accession number A70573; S.coe.: 141 amino acid protein from Streptomyces coelicolor, accession number CAB62687; P.aer.: 138 amino acid protein from Pseudomonas aeruginosa, accession number AAG05547, B.sub.: 140 amino acid protein from Bacillus subtilis, accession number B69824. To predict the domain architecture of the proteins their sequence was searched against the protein domain families data base Pfam (Sanger Centre, UK [http://www.sanger.ac.ulc/Software/Pfam/]). The positions of two predicted CBS domains (Accession number PF00571) are indicated above the alignment.

### BRIEF DESCRIPTION OF THE SEQUENCES

SEQ ID NO:1 shows the nucleotide sequence of Rv3133c from the H37Rv strain of Mycobacterium tuberculosis.

SEQ ID NO:2 shows the amino acid sequence of Rv3133c from the H37Rv strain of Mycobacterium tuberculosis.

SEQ ID NO:3 shows the nucleotide sequence of Rv2623 from the H37Rv strain of Mycobacterium tuberculosis.

SEQ ID NO:4 shows the amino acid sequence of Rv2623 from the H37Rv strain of Mycobacterium tuberculosis.

SEQ ID NO:5 shows the nucleotide sequence of Rv2626c from the H37Rv strain of Mycobacterium tuberculosis.

SEQ ID NO:6 shows the amino acid sequence of Rv2626c from the H37Rv strain of Mycobacterium tuberculosis.

SEQ ID NO:7 shows the nucleotide sequence of Rv3132c from the H37Rv strain of Mycobacterium tuberculosis.

SEQ ID NO: 8 shows the amino acid sequence of Rv3132c from the H37Rv strain of Mycobacterium tuberculosis.

SEQ ID NO:9 shows the amino acid sequence of a variant of Rv2626c from Streptomyces coelicolor.

SEQ ID NO:10 shows the amino acid sequence of a variant of Rv2626c from Pseudomonas aeruginosa.

SEQ ID NO:11 shows the amino acid sequence of a variant of Rv2626c from Bacillus subtilis.

### DETAILED DESCRIPTION OF THE INVENTION

Proteins

The present invention relates to dormancy-induced Mycobacterium proteins. Rv3133c is shown in SEQ ID NO:1 and SEQ ID NO: 2, Rv2623 is shown in SEQ ID NO: 3 and SEQ ID NO: 4 and Rv2626c is shown in SEQ ID NO: 5 and SEQ ID NO: 6. Sequence information for the dormancy-induced mycobacterial proteins of the invention is taken from the complete genome sequence of the H37Rv strain of Mycobacterium tuberculosis (Cole et al., (1998) Nature 393: 537-544) and can be found at http://genolist.pasteur.fr/TubercuList/.

Proteins of the invention may be in a substantially isolated form. It will be understood that the protein may be mixed with carriers or diluents which will not interfere with the intended purpose of the polypeptide and still be regarded as substantially isolated. In particular an isolated protein of the invention will be separated from other Mycobacterium proteins. For example, an isolated polypeptide may be obtained by separating the polypeptide from other Mycobacterial proteins on a 2D-gel and extracting the polypeptide from the gel. A protein of the invention may also be in a substantially purified form, in which case it will generally comprise the polypeptide in a preparation in which more than 50%, e.g. more than 80%, 90%, 95% or 99%, by weight of the protein in the preparation is a protein of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The term “variant” refers to a homologues of Rv3133c, Rv2623 or Rv2626c derived from other strains of Mycobacterium such as the attenuated BCG Pasteur ATCC 35734 strain, or from, other microorganisms such as bacteria including Streptomyces, Pseudomonas and Bacillus.

The term “variant” refers to a protein which is sufficiently similar to Rv3133c, Rv2623 or Rv2626c that the variant is capable of eliciting an immune response to Rv3133c, Rv2623 or Rv2626c. Preferably the variant can be used to raise antibodies to Rv3133c, Rv2623 or Rv2626c. More preferably the variant can be used to generate CD8T-cells that respond to Rv3133c, Rv2623 or Rv2626c.

Preferably a varaint of Rv3133c is capable of binding DNA. More preferably a variant of Rv3133c is capable of binding a sensor histidine protein kinase such as Rv3132c. A variant of Rv3133c preferably has transcriptional regulatory activity.

Typically, polypeptides with more than about 65% identity preferably at least 80% or at least 90% and particularly preferably at least 95% at least 97% or at least 99% identity, with the amino acid sequences of SEQ ID NO: 2, SEQ ID NO: 4 or SEQ ID NO: 6 are considered as variants of the proteins. Such variants include allelic variants and the deletion, modification or addition of single amino acids or groups of amino acids within the protein sequence, as long as the polypeptide maintains the ability to generate an immune response which is effective against Rv3133c, Rv2623 or Rv2626c proteins.

Amino acid substitutions may be made, for example from 1, 2 or 3 to 10, 20 or 30 substitutions. The modified protein generally retains activity as an immunogen. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | NQ |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100, 150, 200, 300, 400 or 500 amino acids in length is considered to fall within the scope of the invention as long as it is capable of eliciting antibodies or a CD8 T-cell response to Rv3133c, Rv2623 or Rv2626c.

Proteins for use in the invention may be chemically modified, e.g. post-translationally modified. For example, they may be glycosylated or comprise modified amino acid residues. They may also be modified by the addition of histidine residues to assist their purification.

Polynucleotides

The invention also includes nucleotide sequences for use in methods of the invention that encode for Rv3133c, Rv2323 or Rv2626c or variant or fragment of any thereof as well as nucleotide sequences which are complementary thereto. The nucleotide sequence may be RNA or DNA. Nucleotide sequence information is provided in SEQ ID NO: 1, SEQ ID NO: 3 or SEQ ID NO: 5. Such nucleotides can be isolated from cells or synthesised according to methods well known in the art, as described by way of example in Sambrook et al, 1989.

Typically a polynucleotide for use in the invention comprises a contiguous sequence of nucleotides which is capable of hybridizing under selective conditions to the coding sequence or the complement of the coding sequence of SEQ ID NO:1, SEQ ID NO: 3 or SEQ ID NO: 5.

A polynucleotide for use in the invention can hydridize to the coding sequence or the complement of the coding sequence of SEQ ID NO:1, SEQ ID NO: 3 or SEQ ID NO: 5 at a level significantly above background. The signal level generated by the interaction between a polynucleotide of the invention and the coding sequence or complement of the coding sequence of SEQ ID NO: 1, SEQ ID NO: 3 or SEQ ID NO: 5 is typically at least 10 fold, preferably at least 100 fold, as intense as interactions between other polynucleotides and the coding sequence of SEQ ID NO: 1, SEQ ID NO: 3 or SEQ ID NO: 5. The intensity of interaction may be measured, for example, by radiolabelling the probe, e.g. with ³²P Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2×SSC at 60° C. up to 65° C. If lower stringency is required suitable conditions include 2×SSC at 60° C.

The coding sequence of SEQ ID NO: 1, SEQ ID NO: 3 or SEQ ID NO: 5 may be modified by nucleotide substitutions, for example from 1, 2 or 3 to 10, 25, 50 or 100 substitutions. The polynucleotide of SEQ ID NO: 1, SEQ ID NO: 3 or SEQ ID NO: 5 may alternatively or additionally be modified by one or more insertions and/or deletions and/or by an extension at either or both ends. The modified polynucleotide generally encodes a polypeptide which has immunogenic activity. Alternatively, a polynucleotide encodes a DNA-binding portion of Rv3133c or a fragment of Rv3133c capable of binding a sensor histidine protein kinase such as Rv3132c. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1, SEQ ID NO: 3 or SEQ ID NO: 5 will generally have at least 60%, at least 70%, at least 80%, at least 90%, at least 95%, at least 98% or at least 99% sequence identity to the coding sequence of SEQ ID NO: 1, SEQ ID NO: 3 or SEQ ID NO: 5 over a region of at least 20, preferably at least 30, for instance at least 40, at least 60, more preferably at least 100 contiguous nucleotides or most preferably over the full length of SEQ ID NO: 1, SEQ ID NO: 3 or SEQ ID NO: 5.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff(1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The polynucleotides have utility in production of the proteins according to the invention, which may take place in vitro, in vivo or ex vivo. The nucleotides may be involved in recombinant protein synthesis or indeed as therapeutic agents in their own right, utilised in nucleic acid vaccines or gene therapy techniques. Nucleotides complementary to those encoding Rv3133c, Rv2323 or Rv2626c, or antisense sequences, may also be used in gene therapy.

Vectors

The present invention also utilizes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Preferably, a polynucleotide for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

Cells

The invention also utilises cells that have been modified to express Rv3133c, Rv2323 or Rv2626c or a variant thereof. Such cells include transient, or preferably stable higher eucaryotic cell lines, such as mammalian cells or insect cells, using for example a baculovirus expression system, lower eukaryotic cells, such as yeast or prokaryotic cells such as bacterial or mycobacterial cells. Particular examples of cells which may be modified by insertion of vectors encoding a polypeptide according to the invention include mammalian HEK293T, CHO, HeLa, BHK, 3T3 and COS cells.

Antibodies

According to another aspect, the present invention also relates to antibodies specific for a protein that is up-regulated under hypoxic conditions or in stationary phase culture and in particular antibodies that are specific for a protein selected from Rv3133c, Rv2623 and Rv2626c. Such antibodies are for example useful in purification, isolation or screening methods involving immunoprecipitation techniques or, indeed, as therapeutic agents in their own right.

Antibodies may be raised against specific epitopes of the proteins according to the invention. Such antibodies may be used to block Rv3133c, or a variant thereof, binding to DNA or to a sensor histidine protein kinase such as Rv3132c. An antibody, or other compound, “specifically binds” to a protein when it binds with preferential or high affinity to the protein for which it is specific but does substantially bind, or binds with only low affinity, to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo or in vitro imaging of Rv3133c, Rv2623 or Rv2626c, for example, in a method of diagnosis.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Monitoring Activity

An important aspect of the present invention is the use of proteins that are upregulated under hypoxic conditions or in stationary phase culture in screening methods. The screening methods may be used to identify substances that bind to hypoxic growth phase, hypoxic stationary phase or non-oxygen limiting stationary phase induced proteins and in particular which bind to Rv3133c, Rv2623 or Rv2626c. Screening methods may also be used to identify agonists or antagonists which may modulate Rv3133c activity, inhibitors or activators of Rv3133c transcriptional activity, and/or agents which up-regulate or down-regulate Rv3133c, Rv2623 or Rv2626c expression.

Any suitable format may be used for the assay. In general terms such screening methods may involve contacting a dormancy-induced or a stationary-phase induced protein with a test agent and monitoring for binding of the test agent to the protein. The protein may be incubated with a test agent. Modulation of Rv3133c activity may be determined. In a preferred aspect, the assay is a cell-based assay. Preferably the assay may be carried out in a single well of a microtitre plate. Assay formats which allow high throughput screening are preferred.

Modulator activity can be determined by contacting cells expressing a dormancy-induced or a stationary-phase induced protein with a substance under investigation and by monitoring an effect mediated by the protein. The protein may be naturally or recombinantly expressed. Preferably, the assay is carried out in vitro using cells expressing recombinant protein. Preferably, control experiments are carried out on cells which do not express the protein of interest to establish whether the observed responses are the result of activation of the protein. Typically the cells are transfected with a Mycobacterium and grown in oxygen defficient conditions. More preferably, the cells are mycobacterial cells grown in conditions suitable for hypoxic growth, conditions that induce the hypoxic stationary phase or conditions that induce the stationary phase in the presence of oxygen.

The binding of a test agent to a dormancy-induced or a stationary-phase induced protein can be determined directly. For example, a radiolabelled test agent can be incubated with a dormancy-induced or a stationary-phase induced protein and binding of the test agent to the protein can be monitored.

Agents that inhibit the interaction of Rv3133c or a variant thereof with DNA or with a sensor histidine protein kinase may also be identified through a yeast 2-hybrid assay or other protein interaction assay such as a co-immunoprecipitation or an ELISA based technique.

Assays may be carried out using cells expressing Rv3133c, and incubating such cells with the test agent optionally in the presence of a sensor histidine protein kinase Rv3132c binding of Rv3133c to the sensor histidine protein kinase may be determined. Alternatively, binding of Rv3133c to DNA or transcriptional activity may be monitored. The results of the assay are typically compared to the results obtained using the same assay in the absence of the test agent.

Monitoring Expression

Assays may also be carried out to identify agents which modify Rv3133c, Rv2623 or Rv2626c expression, for example agents which up- or down-regulate expression. Such assays may be carried out for example by using antibodies for Rv3133c, Rv2623 or Rv2626c to monitor levels of expression following induction of dormant condition. Alternatively, expression may be monitored by determining the effect of the test agent on Rv3133c, Rv 2623 or Rv2626c mRNA levels. Preferably, mRNA levels are monitored in a Mycobacterium grown in oxygen-deprived conditions.

Additional control experiments may be carried out.

Test Agents

Suitable test agents which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alcylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

Diagnostic Agent

Diagnostic methods for the detection of nucleic acid molecules, such as mRNA, encoding a polypeptide of the invention may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Medical Uses

Another aspect of the present invention is the use of the agents that have been identified by screening techniques referred to above in the treatment of mycobacterial infection. The treatment may be therapeutic or prophylactic. The condition of a patient suffering from a mycobacterial infection can thus be improved. The patient may be a human or animal subject. Generally the animal subject is a mammal, typically one which can be naturally or artificially infected by a Mycobacterium. The human or animal subject may be a primate, cow or badger. The subject is typically one which can be naturally or artificially infected by a Mycobacterium. The subject may be at risk of a mycobacterial infection, typically because it is resident in a location in which mycobacterial infection is endemic. The subject may be susceptible to mycobacterial infection due to malnutrition or infection by other pathogens, such as HIV. The Mycobacterium is typically pathogenic and capable of infecting mammals, such as those mammals discussed above. The Mycobacterium is typically M. tuberculosis, M. marinum, M. kansasii, M. bovis or M. avium.

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference. A vaccine composition typically also comprises an adjuvant. The adjuvant may target the peptide to antigen presenting cells (APCs) or to compartments in the antigen processing pathway, for example acting as a carrier protein. The sequence may stimulate a T helper response, such as a response that favours a CD8 T cell response, and thus may comprise a T helper (e.g. Th1) cell epitope. Preferred substances which enhance immunogenicity include sequence from the hepatitis B core antigen, sequence from a stress protein or sequence from Clostridium tetani neurotoxin fragment C. The stress protein is typically a bacterial (e.g. mycobacterial) heat shock protein (HSP) or a protein which has homology with such a protein, such as mycobacterial or E. coli proteins of the HSP 60 and HSP 70 families (e.g. HSP 65 or HSP 71 of mycobacteria) or mammalian homologue (e.g. gp96 of mice or humans, Anthony et al. (1999) Vaccine 17, 373-83).

The substance may cause the polypeptide or vector to adopt a particulate form. The substance may be a virus or virus-like particle (such as a yeast Ty particle, e.g. as in Allsopp et al (1996) Eur. J. Immunol. 26, 1951-9). The substance may be a cytokine, such as a cytokine which stimulates a MHC class I restricted T cell response or favourable MHC class II restricted T cell response (e.g. IL-2, IL-7, IL-12, IFN or GMCSF). The substance may be, for example, CFA, a muramyl dipeptide (e.g. of a mycobacterial cell wall), monophosphoryl lipid A, lipopolysaccharide (e.g. from B. abortus), liposomes, SAF-1, a saponin (e.g. Quil A), keyhole limpet hemocyanin, beta 2-microglobulin, mannan (e.g. oxidised mannan), an acrylic based microbead, or an emulsion (e.g. oil in water or water in oil) such as soybean emulsion (e.g as in Hioe et al. (1996) Vaccine 14, 412-8).

The substances may be administered by enteral or parenteral routes such as via oral, buccal, anal, pulmonary, intravenous, intra-arterial, intramuscular, intraperitoneal, topical or other appropriate administration routes.

The particular route of administration used may aid the stimulating of a CD8 T cell response, and thus the polypeptide vector may be provided in a form suitable for administering by such a route. Delivery by an intramuscular route or by biolistic means is preferred.

A therapeutically effective amount of a modulator is administered to a patient suffering from a mycobacterial infection. An effective amount of a polypeptide of the invention, or a fragment thereof, capable of generating an immune response is administered to a subject at risk of a mycobacterial infection. Stimulation of an immune response can typically be monitored by detecting antibodies directed against Rv3133c, Rv2623 or Rv2626c. Antibodies specific for such proteins may be detected using an ELISAassay.

The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

A vaccine composition is preferably administered in a single dose. One or more, for example, two, three or four, further doses may be required for long term protection against a Mycobacterium infection. Further doses may be against a Mycobacterium infection. Further doses may be administered after a period of from 1 month to 15 years after the initial dose, for example, 1, 2, 3, 4, 5, 8, 10, 12 or 15 years. Several further doses may be administered at intervals after the initial dose, for example at 3, 5, 10 or 15 year intervals.

Nucleic acid encoding a polypeptide of the invention may be administered to a mammal as a nucleic acid vaccine. Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Generally a low dose of antigen favours the development of a CD8 T cell response. Thus a suitable low dose of the polypeptide or vector may be administered to prevent mycobacterial infection. The polypeptide or vector may thus be in an amount and concentration that is suitable for administering to provide an appropriate low dose. In a preferred embodiment the vector is administered in the form of “naked DNA”.

The following Examples illustrate the invention.

### EXAMPLE 1

### Detection and Identification of Dormancy-Induced Proteins

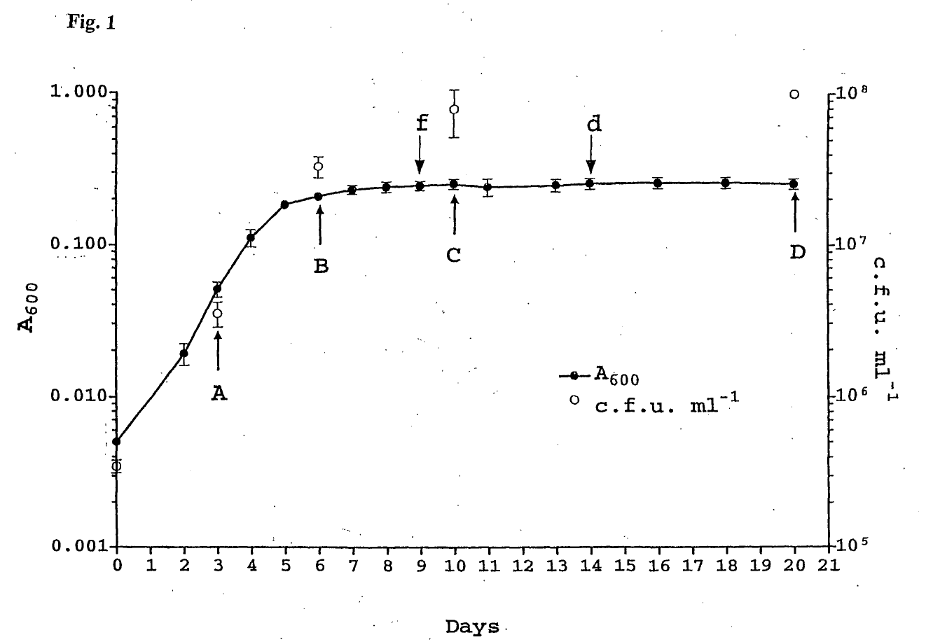

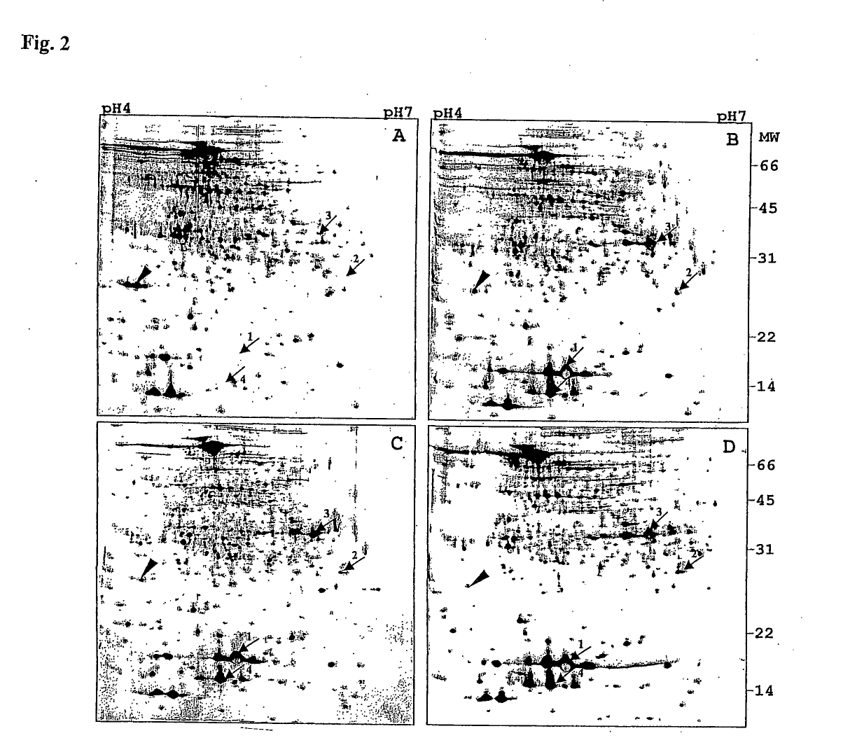

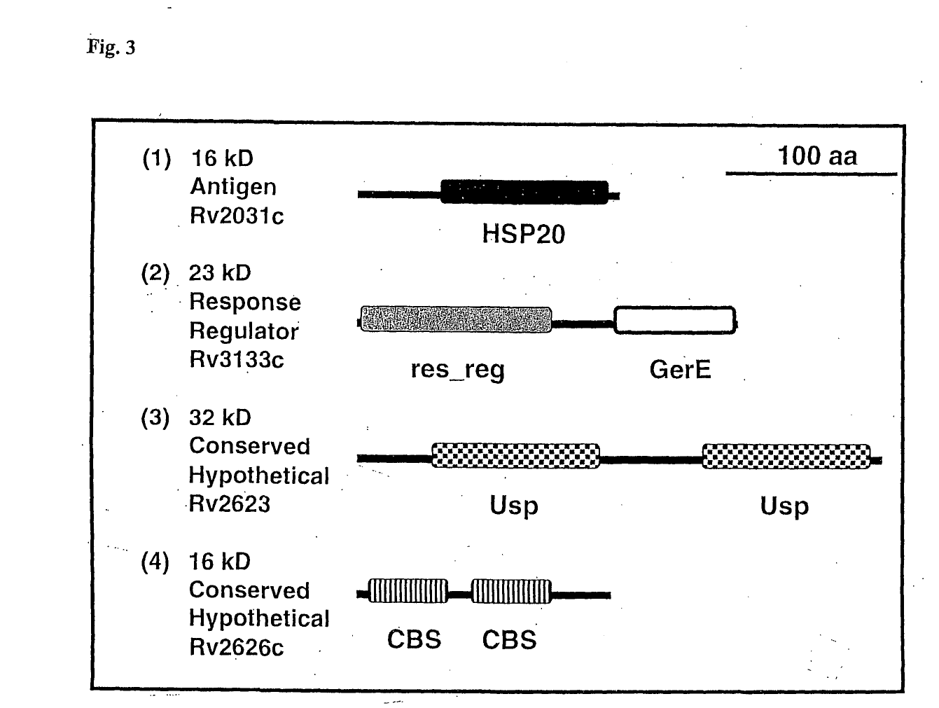

BCG were grown in the Wayne dormancy culture system. Bacilli were harvested at various time points (FIG. 1, arrows A-D), washed twice in phosphate-buffered saline, resuspended in lysis buffer (9 M urea, 4% CHAPS, 50 mM DTT, pefabloc [1 mg ml⁻¹], pepstatin [1 μg ml⁻¹], leupeptin [1 μg ml⁻¹] and disrupted with 0.5 mm glass beads using a Mini Bead Beater (Biospec). Protein concentrations were determined using the BioRad protein assay reagents and protocols. 100 μg of total protein were subjected to isoelectric focusing using pH 4-7 Immobiline Dry Strips and an IPGphor isoelectric focusing unit as recommended by the manufacturer (Amersham Pharmacia) for 62000 Vhrs. For the separation in the second dimension 12.5% sodium dodecyl sulfate polyacrylamide gels were used (Protean IIxi system, BioRad) and proteins were detected by silver staining. FIG. 2. shows a representative set of two-dimensional gels. Four proteins showed a drastic increase in their steady state level in the hypoxic stationary phase (FIG. 2, arrows 1-4). The proteins were not (arrow 1, 2, 4) or only weakly (arrow 3) detectable in the extracts from exponentially growing cultures (FIG. 2A) and appeared as major spots immediately upon oxygen-starvation induced termination of growth (FIG. 2B). Elevated levels of the proteins were maintained throughout the hypoxic stationary phase (FIG. 2C, D). To identify the dormancy-induced proteins, the excised gel spots were subjected to in-gel digestion with trypsin to recover the peptides (Shevchenko et al. (1996) Anal. Chem. 68: 850-858). Peptide sequence tags were generated from selected peptides by nanoelectrospray tandem mass spectrometry using a quadrupole/time-of-flight hybrid instrument (QSTAR, PE Sciex). Protein identity was revealed by searching sequence databases with a combination of the peptide sequence tags and the mass information. Protein 1 was the 16 kD antigen Rv2031c previously reported to be induced in oxygen-starved cultures of tubercle bacilli (Yuan et al. (1996) J. Bacteriol. 178: 4484-4492). Protein 2 was the 23 kD response regulator Rv3133 cc. Proteins 3 and 4 were the 32 kD conserved hypothetical protein Rv2623 and the 16 kD (14 kD on SDS PAGE) conserved hypothetical protein Rv2626c, respectively (FIG. 3). Protein names and Rv numbers are according to the M. tuberculosis H37Rv genome annotation (Cole et al. (1998) Nature 393: 537-544).

### EXAMPLE 2

### Transcript Levels of Dormancy-Induced Proteins

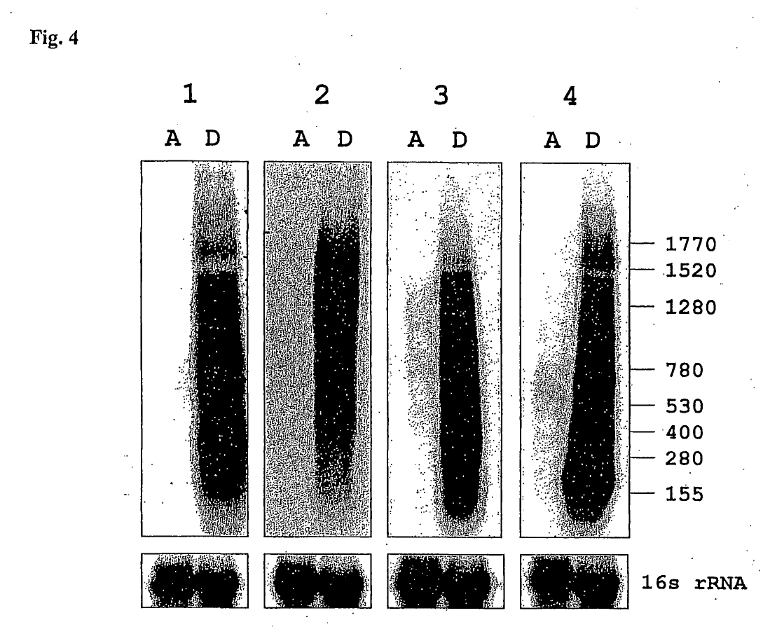

To determine whether the increase of the steady state level of the dormancy-induced proteins correlates with an increase in the steady state level of their mRNAs, total RNA was isolated from exponentially growing and hypoxic stationary phase cultures and subjected to Northern blot analysis as described (Hutter and Dick (1999) FEMS Microbiol. Lett. 178; 63-69). Probes were isolated by PCR using BCG genomic DNA as template. The primers were derived from the M. tuberculosis H37Rv genome sequence and were as follows: 1, Rv2031c (GCCACCACCCTTCCCGTTCAG, ATGTCGTCCTCGTCAGCACCTACC); 2, Rv3133 cc (TCGTAGGTGTAGGCGGGTTC, CGGCGATCTGCTTGTTGGT); 3, Rv2623 (GGCAGCCGTTCCCACATTG, GGCTGATCGCGCACCACCAC); 4, Rv2626c (CCACCGCACGCGACATCAT, CGGAACACGGCGGACCTG); 16S rRNA (GCCTGGGAAACTGGGTCTAA, TCTCCACCTACCGTCAATCC). The identity of the PCR fragments was confirmed by sequencing using a Perkin-Elmer ABI Prism 377 automated sequencer. FIG. 4 shows high levels of the transcripts for all four proteins in dormant bacilli. In exponential growing culture the transcripts were not detectable or were only weakly detectable. This result indicates that the expression of the proteins is regulated at the transcriptional level.

The up-regulation of the steady state level of the 16 kD antigen mRNA in oxygen-starved dormant culture is in apparent contradiction to the down-regulation of the transcript in nonpagitated cultures of the tubercle bacillus observed by Hu et al. (1999) J. Bacteriol. 181: 1380-1387. The 16 kD antigen had been previously reported to be induced in oxygen-starved stationary phase cultures of tubercle bacilli. The protein belongs to the 20 kD small heat shock protein family (FIG. 3) and was shown to possess chaperon function ascribed to other members of the family. The three new dormancy-induced proteins were predicted by the M. tuberculosis H37Rv genome project (Cole et al.). However, biochemical or genetic data for these proteins are not available. Two of the three new dormancy-induced proteins are annotated as ‘conserved hypothetical proteins’ (Cole et al. (1998) Nature 393: 537-544). A similarity search against the protein domain families (pfam) database (1) suggested that the 32 kD conserved hypothetical protein shown in SEQ ID NO: 4 contains two ‘Universal Stress Protein’ (Usp) domains (FIG. 3). This domain is found in the UspA protein in E. coli. UspA is up-regulated in stationary phase cultures and plays a role in the survival of growth-arrested cells (Diez et al. (2000) Mol. Micobiol. 36: 1494-1503). The 16 kD conserved hypothetical protein shown in SEQ ID NO: 6 is predicted to contain two ‘Cystathionine-beta synthase’ (CBS) domains (FIG. 3). CBS domains are found in functionally diverse proteins. A function for this domain is not known. Most intriguing is the dormancy-dependent up-regulation of the 23 kD response regulator shown in SEQ ID NO: 2. This response regulator contains a ‘helix-turn-helix’ DNA binding domain and is thus likely to act as a phosphorylation-dependent transcription factor (FIG. 3). Response regulators, together with their respective sensor histidine protein kinase, are part of two-component signal transduction systems and play key roles in a variety of developmental and adaptive processes in bacteria. Thus, it is conceivable that the 23 kD response regulator plays a role in the control of the mycobacterial dormancy response. Inspection of the genomic locus surrounding the gene encoding the 23 kD response regulator in M. tuberculosis H37Rv showed that the gene appears to form an operon with the gene Rv3132c annotated as putative sensor histidine protein kinase (Cole et al). The genomic organisation is identical in BCG and could indicate that this putative kinase represents the sensor involved in the control of the activity of the dormancy-induced 23 kD response regulator.

### EXAMPLE 3

### Detection and Identification of Stationary Phase—Induced Proteins

M. bovis BCG were grown at 37° C. in Dubos Tween-albumin broth (BD Biosience). Bacilli were sub-cultured once in liquid medium until they reached an early exponential growth phase (A₈₀₀=0.2-0.3) before inoculation to an experimental culture. To grow experimental cultures medium was dispensed in 100 ml aliquots to roller bottles, 10×14 cm, and pre-cultures were diluted to A600=0.05 (5×10⁶ cfu/ml). Cultures were aerated by incubation on a roller apparatus at 1 rpm. The roller bottles were opened daily for turbidity measurements and to allow exchange of the air. Growth was monitored by measuring the optical density of the cultures in. a Ultrospec 3000 spectrophotometer (Pharmacia).

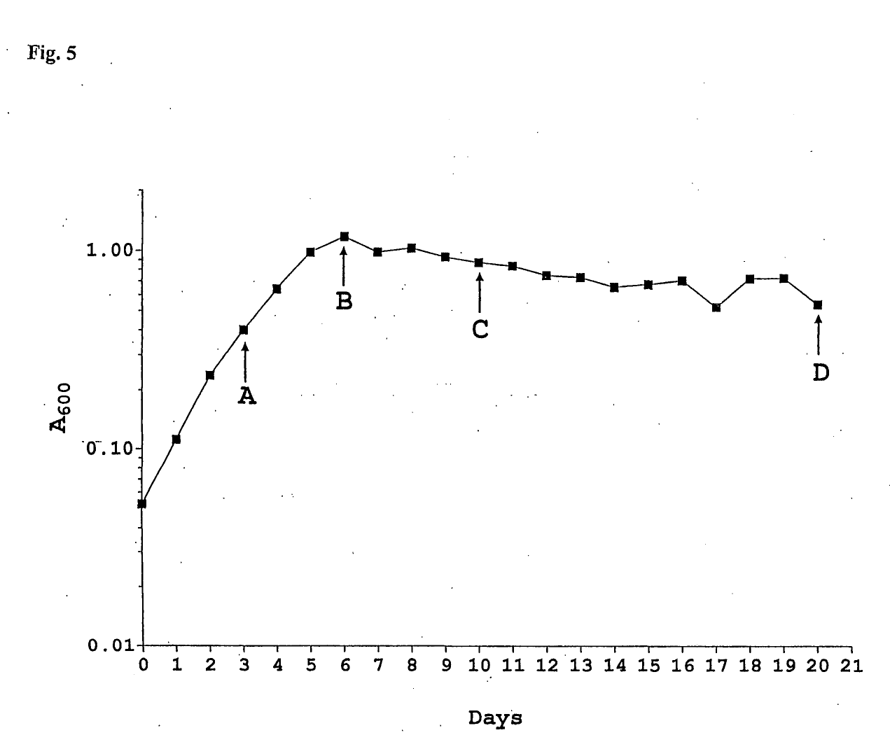

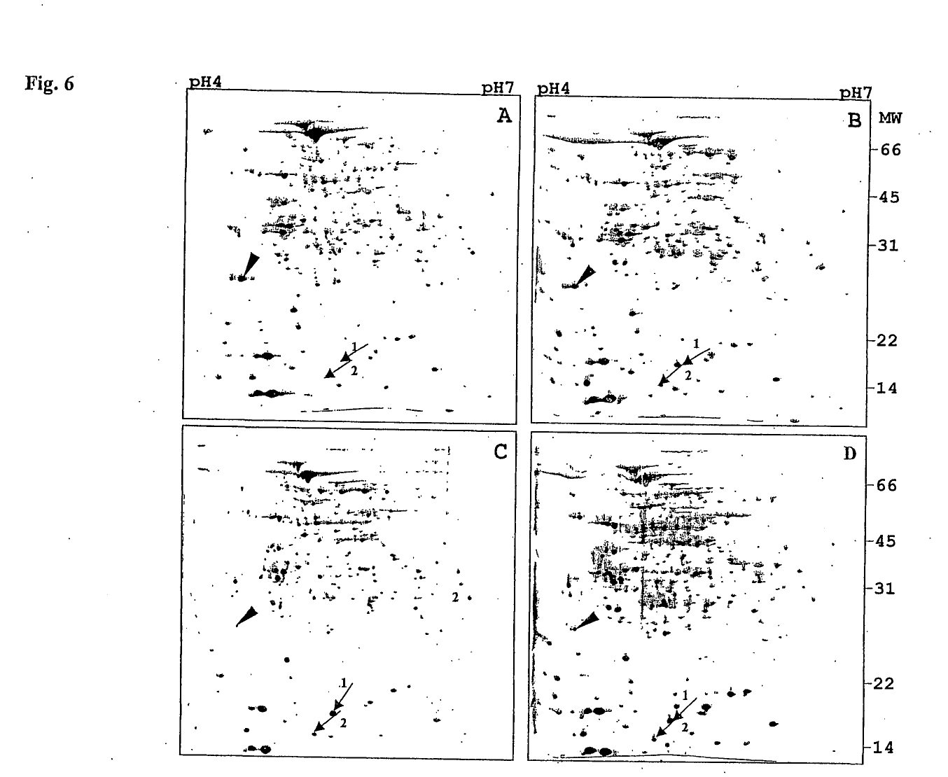

Protein extracts were prepared from different time points corresponding to the exponential phase, entry into stationary phase, early and late stationary phase (FIG. 5, arrows A-D). For the preparation of protein extracts the cells were washed twice in phosphate-buffered saline, resuspended in lysis buffer (9 M urea, 4% CHAPS, 50 mM DTT, pefabloc [1 mg ml⁻¹], pepstatin [1 μg ml⁻¹],.leupeptin [1 μg m;⁻¹]) and disrupted with 0.5 mm glass beads using a Mini Bead Beater (Biospec). Protein concentrations were determined using the BioRad protein assay reagents and protocols. 100 μg of total protein was subjected to isoelectric focussing using pH 4-7 Immobiline Dry Strips and a IPGphor isoelectic focusing unit as recommended by the manufacture (Amersham Pharmacia) for 62000 Vhrs. For the separation in the second dimension 12.5% sodium dodecyl sulfate polyacrylamide gels were used (Protean IIxi system, BioRad). Proteins were detected by silver staining and the gels were evaluated by visual inspection. FIG. 6 shows a representative set of two-dimensional gels. Two proteins of 16 kD and 14 kD showed a drastic increase in their steady state level in the stationary phase (FIG. 6, arrows 1 and 2). The two proteins were not detectable in the extracts from expoentially growing cultures (FIG. 6A) and appeared as major spots immediately upon termination of growth (FIG. 6B). Elevated levels of the proteins were maintained throughout the stationary phase (FIG. 6C, D). The 16 kD protein is the BCG counterpart of the 16 kD antigen (Rv2031c). The 14 kD polypeptide is the counterpart of the protein Rv2626c predicted by the M tuberculosis H37Rv genome project.

The marked up-regulation of the 16 kD antigen and the 14 kD protein immediately upon termination of growth suggests that they play roles in the entry into the stationary phase and/or in maintaining viability of stationary phase bacilli. The 16 kD antigen has been previously reported to be induced in stationary phase cultures of tubercle bacilli (Yuan et al. (1996) J. Bacteriol. 178: 4484-4492).

### EXAMPLE 4

### Homologues of the Stationary Phase-Induced and Dormancy-Induced Rv2626c Protein

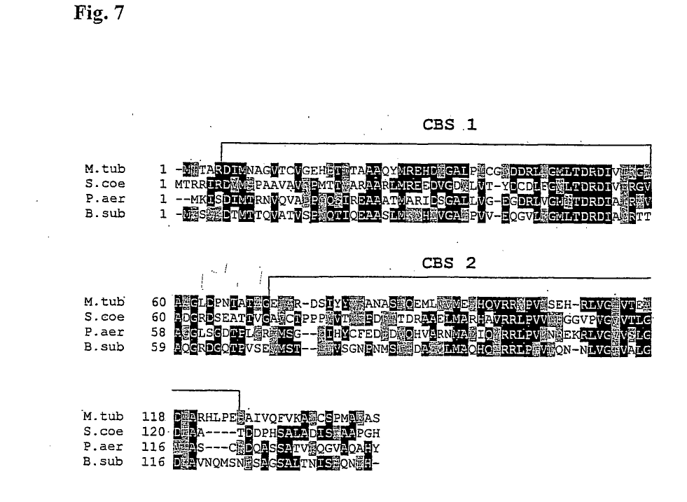

Biochemical or genetic data for the new stationary phase-induced 14 kD protein/dormancy-induced 16 kD protein shown in SEQ ID NO:6 are not available. A similarity search against the protein domain families (pfam) database suggested that the protein consists of a pair of CBS domains (FIGS. 3 and 7). The recently discovered CBS domain is named after Cystathionine Beta Synthase where it was originally identified. The domain is usually present as a pair and this CBS domain-dimer associates to form a single compact structure. Pairs of CBS domains are found in a large number of functionally diverse proteins such as inosine-monophosphate dehydrogenases and chloride channels. Although the role of the CBS domain in these proteins is unclear, it may be involved in protein-protein interaction and protein regulation. In contrast to these proteins that contain a pair of CBS domains in the context of other, unrelated domains, the small stationary phase-induced mycobacterial protein appears to consist of only a pair of CBS domains, not fused to other domains (FIGS. 3 and 7). It is interesting to note that small molecular weight proteins with the same predicted domain architecture and about 35% sequence identity can be found in other bacteria such as Streptomyces coelicolor, Pseudomas aeruginoas and Bacillus subtilis (FIG. 7). This suggests that the Rv2626c dormancy-induced and stationary phase-induced mycobacterial protein is a member of a new bacterial protein family.

### EXAMPLE 5

### Overexpression, Purification and Phosphorylation of the 23 kD Response Regulator Protein

The dormancy-induced response regulator Rv3133c is predicted to function as a phosporylation-dependent transcription regulator. Thus, it is likely that the protein binds to target promoters and recruits RNA polymerase to initiate transcription of essential dormancy genes. Hence, prevention of DNA binding by inhibitors should abolish response regulator function. Under the assumption that a cognate promoter sequence is available, an in vitro DNA binding assay can be carried out to screen for inhibitors of the response regulator's DNA binding activity:

Using gene specific primers derived form the M. tuberculosis H37Rv genome sequence (Cole et al. (1998)) the coding sequence of the Rv3133c gene is isolated by PCR and a glutathione S-transferase fusion protein is constructed, overexpressed and purified as described in Ulijasz et al. (2000), Biochemistry 39, 11427-11424 and Sambrook et al. (1989) Molecular cloning, Cold Spring Habor Laboratory Press, Cold Spring Habor, N.Y. The purified protein is phosphorylated as described in Ulijasz et al. using 50 mM lithium acetyl phosphate.

### EXAMPLE 6

### Isolation and Radioactive Labelling of the Promoter DNA

The cognate promoter DNA is PCR amplified using primers derived from the M tuberculosis H37Rv genome sequence (Cole et al. (1998)) and 5′-end labelled. with y-³²P-ATP and T4 polynucleotide kinase according to standard methods as described in Sambrook et al. (1989).

### EXAMPLE 7

### Binding of the Phosphorylated Response Regulator to its Cognate Promoter and Gel Shift Analysis of the Binding

Protein-DNA complexes are formed in a total volume of 15 μl which contain 4 pmol of phosphorylated response regulator which was preincubated with the appropriate inhibitors for 10 min at room temperature in 20 mM HEPES (pH 7.2), 5-mM MgCl₂, 0.1 M Na2EDTA, 0.5 mM CaCl₂, 10% glycerol and 0.5 μg of salmon DNA as described in Ulijasz et al. (2000). To start the reaction, ³²P-labeled promoter DNA (0.65 ng in 1 μl) is added, and the reaction mixture is incubated on ice for 15 min.

The resultant complexes are analyzed by non-denaturing polyacrylamide gel electrophoresis (PAGE). Gels are dried and exposed to a phosphorimager as described in Ulijasz et al. (2000).

An inhibitor of the response regulator will prevent the binding of the protein to its promoter and hence reduce complex formation of the two components. Complex formation, or the absence of complex formation is detectable in the gel shift analysis.

1. A method for the identification of an anti-mycobacterial agent that modulates the activity and/or expression of a protein expressed by a Mycobacterium in non-oxygen limiting stationary phase, hypoxic stationary phase or hypoxic growth phase, which method comprises:
(i) contacting a test agent and a protein selected from RV3133c, Rv2623, Rv2626c, a variant of Rv3133c, Rv2623 or Rv2626c and a fragment of Rv3133c, RV2623, Rv2626c or said variant, or a polynucleotide or expression vector encoding said protein;
(ii) monitoring the effect of the test agent on the activity and/or expression of said protein, thereby determining whether the test agent modulates the activity and/or expression of a protein expressed by a Mycobacterium in non-oxygen limiting stationary phase, hypoxic stationary phase or hypoxic growth phase.

2. A method according to claim 1 wherein step (ii) comprises monitoring binding of said protein to the test agent.

3. A method according to claim 1 wherein step (ii) comprises monitoring binding of Rv3133c, a variant thereof or a fragment of either thereof to DNA.

4. A method according to claim 1 wherein step (ii) comprises monitoring binding of Rv3133c, a variant thereof or a fragment of either thereof to a sensor histidine protein kinase.

5. A method according to claim 4 wherein the sensor histidine protein kinase is Rv3132c, a variant thereof or a fragment of either thereof.

6. A method according to claim 1 wherein step (ii) comprises monitoring the transcriptional activity of a gene regulated by Rv3133c, a variant thereof or a fragment of either thereof.

7. A method for the identification of a diagnostic agent that binds to a protein expressed by a Mycobacterium in non-oxygen limiting stationary phase, hypoxic stationary phase or hypoxic growth phase, or to a polynucleotide encoding said protein, which method comprises:
(i) contacting a test agent and a protein selected from Rv3133c, Rv2623, Rv2626c, a variant of Rv3133c, Rv2623, Rv2626c or a fragment of Rv3133c, Rv2623, Rv2626c or said variant, or a polynucleotide encoding said protein;
(ii) monitoring any interaction between the test agent and said protein or said polynucleotide, thereby determining whether the test agent binds a protein or polynucleotide expressed by a Mycobacterium in non-oxygen limiting stationary phase, hypoxic stationary phase or hypoxic growth phase.

8. A method according to any preceding claim wherein said agent is a variant or fragment of a protein expressed by a Mycobacterium in non-oxygen limiting stationary phase, hypoxic stationary phase or hypoxic growth phase, a polynucleotide which encodes a variant or fragment of said protein or a polynucleotide which hybridises under stringent conditions to a sequence encoding said protein.

9. An agent which is identifiable by a method according to any preceding claim.

10. An agent according to claim 9 which inhibits activity and/or expression of a protein expressed by a Mycobacterium in non-oxygen limiting stationary phase hypoxic stationary phase or hypoxic growth phase.

11. An antibody specific for a protein selected from Rv3133c, Rv2623 or Rv2626c.

12. A pharmaceutical composition comprising a pharmaceutically effective carrier and as an active ingredient an effective amount of an agent according to claim 9 or 10 or an antibody according to claim 11.

13. A vaccine composition comprising as an active ingredient an effective amount of a protein selected from Rv3133c, Rv2623, Rv2626c and a variant of any thereof, or an immunogenic fragment any said protein, and a pharmaceutically effective carrier.

14. An agent according to claim 9 or 10, an antibody according to claim 11, a pharmaceutical composition according to claim 12 or a vaccine composition according to claim 13 for use in a method of treatment of the human or animal body by therapy or in a diagnostic method practised on the human or animal body.

15. Use of an agent according to claim 9 or 10, an antibody according to claim 11, a pharmaceutical composition according to claim 12 or a vaccine composition according to claim 13 in the manufacture of a medicament for the diagnosis, prophylaxis or treatment of a mycobacterial infection.

16. Use according to claim 15 wherein said mycobacterial infection is tuberculosis.

17. A method of treating a subject suffering from a mycobacterial infection, which method comprises administering to said subject a therapeutically effective amount of an agent according to claim 9 or 10, an antibody according to claim 11 or a pharmaceutical composition according to claim 12.

18. A method for preventing a mycobacterial infection in a subject at risk thereof, which method comprises administrating to said subject a prophylactically effective amount of a protein selected from Rv3133c, Rv2626c, Rv2623, a variant of any thereof and a fragment of any said protein, an agent according to claim 9 or 10, an antibody according to claim 11 or a vaccine composition according to claim 13.

19. A method for detecting a mycobacterial infection in a sample, which method comprises detecting the presence of a protein expressed by a Mycobacterium in non-oxygen limiting stationary phase, hypoxic stationary phase or hypoxic growth phase or a nucleic acid encoding said protein in said sample, wherein said protein is selected from Rv3133c, Rv2623, RV2626c and a variant of any thereof.

20. A method according to claim 19 which comprises:
(i) contacting a sample and an agent according to claim 9 or an antibody according to claim 11; and
(ii) monitoring binding of said agent or antibody to said sample, thereby determining whether said sample comprises a protein expressed by a Mycobacterium in non-oxygen limiting stationary phase, hypoxic stationary phase or hypoxic growth phase, or a nucleic acid encoding said protein and hence whether said sample is infected with a Mycobacterium.

21. A method according to claim 20 wherein said nucleic acid is RNA.

22. An in vitro method of diagnosing a mycobacterial infection in a subject, which method comprises a method according to any one of claims 19 to 21 and wherein said sample is a sample from said subject.

23. An in vitro or in vivo method for diagnosing a mycobacterial infection in a subject which method comprises monitoring expression of a protein expressed by a Mycobacterium in non-oxygen limiting stationary phase, hypoxic stationary phase or hypoxic growth phase, wherein said protein is selected from Rv3133c, Rv2623, RV2626c and a variant of any thereof.

24. A method according to claim 23, which method comprises administering to a subject at risk of mycobacterial infection an agent according to claim 9 or an antibody according to claim 11 and monitoring the response to the said agent or antibody.

25. A method according to any one of claims 17 to 24 wherein said mycobacterial infection is tuberculosis.

26. A method of obtaining a protein selected from Rv3133c, Rv2623, Rv2626c and a variant thereof, which method comprises maintaining a Mycobacterium under aerobic or anaerobic conditions suitable for inducing non-oxygen limiting stationary phase, hypoxic stationary phase or hypoxic growth phase expressed proteins and isolating the said protein.

27. Use of a protein selected from Rv3133c, Rv2623, Rv2626c, a variant of Rv3133c, Rv2623 or Rv2626c and a fragment of Rv3133c, Rv2623, Rv2626c or said variant in a method for the identification of an anti-mycobacterial agent.

28. Use of a protein selected from Rv3133c, Rv2623, Rv2626c, a variant of Rv3133c, Rv2623 or Rv2626c and a fragment of Rv3133c, Rv2623, Rv2626c or said variant in a method for the identification of an agent for diagnosing a dormant mycobacterial infection.

29. A method for the identification of an anti-mycobacterial agent that modulates the activity or expression of a protein expressed by a Mycobacterium in non-oxygen limiting stationary phase, hypoxic stationary phase, or hypoxic growth phase, which method comprises:
(i) contacting a test agent with a protein selected from the group consisting of RV3133c, Rv2623, Rv2626c, a variant of Rv3133c, Rv2623 or Rv2626c and a fragment of Rv3133c, RV2623, Rv2626c or said variant, or a polynucleotide or expression vector encoding said protein; and
(ii) monitoring the effect of the test agent on the activity or expression of said protein, thereby determining whether the test agent modulates the activity or expression of a protein expressed by a Mycobacterium in non-oxygen limiting stationary phase, hypoxic stationary phase, or hypoxic growth phase.

30. A method according to claim 29, wherein step (ii) comprises monitoring binding of said protein to the test agent.

31. A method according to claim 29, wherein step (ii) comprises monitoring binding of Rv3133c, a variant thereof or a fragment of either thereof to DNA.

32. A method according to claim 29, wherein step (ii) comprises monitoring binding of Rv3133c, a variant thereof or a fragment of either thereof to a sensor histidine protein kinase.

33. A method according to claim 32, wherein the sensor histidine protein kinase is Rv3132c, a variant thereof or a fragment of either thereof.

34. A method according to claim 29, wherein step (ii) comprises monitoring the transcriptional activity of a gene regulated by Rv3133c, a variant thereof or a fragment of either thereof.

35. A method for the identification of a diagnostic agent that binds to a protein expressed by a Mycobacterium in non-oxygen limiting stationary phase, hypoxic stationary phase or hypoxic growth phase, or to a polynucleotide encoding said protein, which method comprises:
(i) contacting a test agent and a protein selected from the group consisting of Rv3133c, Rv2623, Rv2626c, a variant of Rv3133c, Rv2623, Rv2626c or a fragment of Rv3133c, Rv2623, Rv2626c or said variant, or a polynucleotide encoding said protein; and
(ii) monitoring any interaction between the test agent and said protein or said polynucleotide, thereby determining whether the test agent binds a protein or polynucleotide expressed by a Mycobacterium in non-oxygen limiting stationary phase, hypoxic stationary phase or hypoxic growth phase.

36. A method according to claim 29, wherein said agent is a variant or fragment of a protein expressed by a Mycobacterium in non-oxygen limiting stationary phase, hypoxic stationary phase or hypoxic growth phase, a polynucleotide which encodes a variant or fragment of said protein or a polynucleotide which hybridises under stringent conditions to a sequence encoding said protein.

37. An agent identified or identifiable by a method according to claim 29.

38. An agent according to claim 37, which inhibits activity or expression of a protein expressed by a Mycobacterium in non-oxygen limiting stationary phase hypoxic stationary phase or hypoxic growth phase.

39. An antibody specific for a protein selected from the group consisting of Rv3133c, Rv2623, and Rv2626c.

40. A pharmaceutical composition comprising a pharmaceutically effective carrier and, as an active ingredient, an effective amount of an agent according to claim 37.

41. An immunogenic composition comprising as an active ingredient an effective amount of a protein selected from the group consisting of Rv3133c, Rv2623, Rv2626c, and a variant of any thereof, or an immunogenic fragment of any said protein, and a pharmaceutically effective carrier.

42. A method of treating a subject suffering from a mycobacterial infection, which method comprises administering to said subject a therapeutically effective amount of an agent according to claim 37.

43. A method for preventing a mycobacterial infection in a subject at risk thereof, which method comprises administrating to said subject a prophylactically effective amount of a protein selected from the group consisting of Rv3133c, Rv2626c, Rv2623, a variant of any thereof and a fragment of any said protein.

44. A method for detecting a mycobacterial infection in a sample, which method comprises detecting the presence of a protein expressed by a Mycobacterium in non-oxygen limiting stationary phase, hypoxic stationary phase, or hypoxic growth phase or a nucleic acid encoding said protein in said sample, wherein said protein is selected from the group consisting of Rv3133c, Rv2623, RV2626c, and a variant of any thereof.

45. A method according to claim 44, which comprises:
(i) contacting a sample and an agent according to claim 37; and
(ii) monitoring binding of said agent to said sample, thereby determining whether said sample comprises a protein expressed by a Mycobacterium in non-oxygen limiting stationary phase, hypoxic stationary phase or hypoxic growth phase, or a nucleic acid encoding said protein and hence whether said sample is infected with a Mycobacterium.

46. A method according to claim 45, wherein said nucleic acid is RNA.

47. An in vitro method of diagnosing a mycobacterial infection in a subject, which method comprises a method according to claim 44 and wherein said sample is a sample from said subject.

48. An in vitro or in vivo method for diagnosing a mycobacterial infection in a subject, which method comprises monitoring expression of a protein expressed by a Mycobacterium in non-oxygen limiting stationary phase, hypoxic stationary phase or hypoxic growth phase, wherein said protein is selected from the group consisting of Rv3133c, Rv2623, RV2626c and a variant of any thereof.

49. A method according to claim 48, which method comprises administering to a subject at risk of mycobacterial infection an agent according to claim 9 and monitoring the response to the said agent.

50. A method according to claim 42, wherein said mycobacterial infection is tuberculosis.

51. A method of obtaining a protein selected from the group consisting of Rv3133c, Rv2623, Rv2626c and a variant thereof, which method comprises maintaining a Mycobacterium under aerobic or anaerobic conditions suitable for inducing non-oxygen limiting stationary phase, hypoxic stationary phase or hypoxic growth phase expressed proteins and isolating said protein.

52. Use of a protein selected from the group consisting of Rv3133c, Rv2623, Rv2626c, a variant of Rv3133c, Rv2623 or Rv2626c and a fragment of Rv3133c, Rv2623, Rv2626c or said variant in a method for the identification of an anti-mycobacterial agent.

53. Use of a protein selected from the group consisting of Rv3133c, Rv2623, Rv2626c, a variant of Rv3133c, Rv2623 or Rv2626c and a fragment of Rv3133c, Rv2623, Rv2626c or said variant in a method for the identification of an agent for diagnosing a dormant mycobacterial infection.

54. A method according to claim 35, wherein said agent is a variant or fragment of a protein expressed by a Mycobacterium in non-oxygen limiting stationary phase, hypoxic stationary phase or hypoxic growth phase, a polynucleotide which encodes a variant or fragment of said protein or a polynucleotide which hybridises under stringent conditions to a sequence encoding said protein.

55. An agent identified or identifiable by a method according to claim 35.

56. An agent according to claim 55, which inhibits activity or expression of a protein expressed by a Mycobacterium in non-oxygen limiting stationary phase hypoxic stationary phase or hypoxic growth phase.

57. A pharmaceutical composition comprising a pharmaceutically effective carrier and as an active ingredient an effective amount of an agent according to claim 55.

58. A pharmaceutical composition comprising a pharmaceutically effective carrier and as an active ingredient an effective amount of an antibody according to claim 39.

59. A method of treating a subject suffering from a mycobacterial infection, which method comprises administering to said subject a therapeutically effective amount of an antibody according to claim 39.

60. A method for preventing a mycobacterial infection in a subject at risk thereof, which method comprises administrating to said subject a prophylactically effective amount of an agent according to claim 37.

61. A method for preventing a mycobacterial infection in a subject at risk thereof, which method comprises administrating to said subject a prophylactically effective amount of an antibody according to claim 39.

62. A method according to claim 44, which comprises:
(i) contacting a sample and an antibody according to claim 39; and
(ii) monitoring binding of said antibody to said sample, thereby determining whether said sample comprises a protein expressed by a Mycobacterium in non-oxygen limiting stationary phase, hypoxic stationary phase or hypoxic growth phase, or a nucleic acid encoding said protein and hence whether said sample is infected with a Mycobacterium.

63. A method according to claim 62, wherein said nucleic acid is RNA.

64. A method according to claim 48, which method comprises administering to a subject at risk of mycobacterial infection an antibody according to claim 11 and monitoring the response to the said antibody.

65. A method according to claim 43, wherein said mycobacterial infection is tuberculosis.

66. A method according to claim 44, wherein said mycobacterial infection is tuberculosis.

67. A method according to claim 45, wherein said mycobacterial infection is tuberculosis.

68. A method according to claim 47, wherein said mycobacterial infection is tuberculosis.

69. A method according to claim 48, wherein said mycobacterial infection is tuberculosis.


SEQUENCE LISTING (21 sequences)

SEQ ID NO: 1 - DNA, 651 bp, Mycobacterium tuberculosis
  CDS (1)...(651)
gtggtaaaggtcttcttggtcgatgaccacgaggtggtgcgtcgtggtctggttgacttgcttggggccgatcccgagcttgacgtcgtaggtgaggcgggttcggtcgccgaggcgatggccagggttcctgccgcgcgcccagatgtcgcggtgctggatgtccggttgcccgatggcaacggcattgaactgtgccgcgatctgttgtcccgcatgcccgatctgcgctgtctgatcctcacgtcctacacctctgacgaggccatgctagatgcgattctcgccggtgccagcggatatgtcgtcaaagacatcaagggaatggagttggcgcgcgccgtcaaagatgtgggcgctggacggtcgctgctggacaatcgggccgcggccgcgctgatggccaagctgcgcggtgccgccgagaagcaggacccgctatcaggccttaccgaccaggagcggacgctactgggcctgcttagcgagggcctgaccaacaagcagatcgccgaccgaatgttcctagccgaaaagacggtgaagaactacgtgtcgcggttgctggccaagctgggcatggaacgtcggacgcaagccgcggtattcgcgacggagttgaagcgctcgcggccacccggtgatggacca

SEQ ID NO: 2 - protein, 217 aa, Mycobacterium tuberculosis
VVKVFLVDDHEVVRRGLVDLLGADPELDVVGEAGSVAEAMARVPAARPDVAVLDVRLPDGNGIELCRDLLSRMPDLRCLILTSYTSDEAMLDAILAGASGYVVKDIKGMELARAVKDVGAGRSLLDNRAAAALMAKLRGAAEKQDPLSGLTDQERTLLGLLSEGLTNKQIADRMFLAEKTVKNYVSRLLAKLGMERRTQAAVFATELKRSRPPGDGP

SEQ ID NO: 3 - DNA, 891 bp, Mycobacterium tuberculosis
  CDS (1)...(891)
atgtcatcgggcaattcatctctgggaattatcgtcgggatcgacgattcaccggccgcacaggttgcggtgcggtgggcagctcgggatgcggagttgcgaaaaatccctctgacgctcgtgcacgcggtgtcgccggaagtagccacctggctggaggtgccactgccgccgggcgtgctgcgatggcagcaggatcacgggcgccacctgatcgacgacgcactcaaggtggttgaacaggcttcgctgcgcgctggtccccccacggtccacagtgaaatcgttccggcggcagccgttcccacattggtcgacatgtccaaagacgcagtgctgatggtcgtgggttgtctcggaagtgggcggtggccgggccggctgctcggttcggtcagttccggcctgctccgccacgcgcactgtccggtcgtgatcatccacgacgaagattcggtgatgccgcatccccagcaagcgccggtgctagttggcgttgacggctcgtcggcctccgagctggcgaccgcaatcgcattcgacgaagcgtcgcggcgaaacgtggacctggtggcgctgcacgcatggagcgacgtcgatgtgtcggagtggcccggaatcgattggccggcaactcagtcgatggccgagcaggtgctggccgagcggttggcgggttggcaggagcggtatcccaacgtagccataacccgcgtggtggtgcgcgatcagccggcccgccagctcgtccaacgctccgaggaagcccagctggtcgtggtcggcagccggggccgcggcggctacgccggaatgctggtggggtcggtaggcgaaaccgttgctcagctggcgcggacgccggtcatcgtggcacgcgagtcgctgact

SEQ ID NO: 4 - protein, 297 aa, Mycobacterium tuberculosis
MSSGNSSLGIIVGIDDSPAAQVAVRWAARDAELRKIPLTLVHAVSPEVATWLEVPLPPGVLRWQQDHGRHLIDDALKVVEQASLRAGPPTVHSEIVPAAAVPTLVDMSKDAVLMVVGCLGSGRWPGRLLGSVSSGLLRHAHCPVVIIHDEDSVMPHPQQAPVLVGVDGSSASELATAIAFDEASRRNVDLVALHAWSDVDVSEWPGIDWPATQSMAEQVLAERLAGWQERYPNVAITRVVVRDQPARQLVQRSEEAQLVVVGSRGRGGYAGMLVGSVGETVAQLARTPVIVARESLT

SEQ ID NO: 5 - DNA, 429 bp, Mycobacterium tuberculosis
  CDS (1)...(429)
atgaccaccgcacgcgacatcatgaacgcaggtgtgacctgtgttggcgaacacgagacgctaaccgctgccgctcaatacatgcgtgagcacgacatcggcgcgttgccgatctgcggggacgacgaccggctgcacggcatgctcaccgaccgcgacattgtgatcaaaggcctggctgcgggcctagacccgaataccgccacggctggcgagttggcccgggacagcatctactacgtcgatgcgaacgcaagcatccaggagatgctcaacgtcatggaagaacatcaggtccgccgtgttccggtcatctcagagcaccgcttggtcggaatcgtcaccgaagccgacatcgcccgacacctgcccgagcacgccattgtgcagttcgtcaaggcaatctgctcgcccatggccctcgccagc

SEQ ID NO: 6 - protein, 143 aa, Mycobacterium tuberculosis
MTTARDIMNAGVTCVGEHETLTAAAQYMREHDIGALPICGDDDRLHGMLTDRDIVIKGLAAGLDPNTATAGELARDSIYYVDANASIQEMLNVMEEHQVRRVPVISEHRLVGIVTEADIARHLPEHAIVQFVKAICSPMALAS

SEQ ID NO: 7 - DNA, 1734 bp, Mycobacterium tuberculosis
  CDS (1)...(1734)
atgacaacagggggcctcgtcgacgaaaacgacggcgccgcaatgcgtccactgcgtcacacgctctcccaactacgcctgcacgagctgctggtcgaggtgcaggaccgggtcgagcagatcgtcgagggccgggaccgcctcgatggtctggtggaggccatgctcgtggtcacagcgggcctggacctggaggcaaccctacgcgctatcgtgcattcagcgaccagccttgtcgatgcgcgctatggcgctatggaggtgcacgaccggcagcatcgggtattgcactttgtctatgaaggcatcgacgaggagaccgttcggcggatcggccacctaccgaaaggcctaggcgtcatcgggctgctcatcgaagatcccaaaccgttacggctggacgatgtttctgcgcacccggcctcgattggttttccgccgtatcatccgccgatgcgtaccttcctcggggtaccggttcgggtgcgcgatgaatcgttcggcactctgtacctgactgacaagaccaacgggcaaccgttcagcgacgacgacgaggttctggtccaggcgctggcggccgccgcgggtatcgcagtcgcgaatgcccggctctaccagcaggctaaggcgcgtcagtcgtggatcgaggccacccgtgacatcgccaccgagttgttgtccggcaccgaacccgcgacggtgttccggcttgtcgccgcggaggcgctcaagctgacggcggctgacgctgccctggtagccgttcccgtcgacgaggacatgcctgccgctgacgtgggggagctgctggtgattgaaacagtcggcagcgctgtggcttccattgttgggcgaacgattccggtggcgggcgcggtgctgcgggaggtcttcgtcaacggcattccgcgacgggtcgaccgggtcgatttggaaggcctggacgaactggccgacgcaggtccggcgctgctgttgccgctgcgggccagaggtaccgtagcgggtgtcgttgttgtgctgagtcaaggcggtccaggggctttcaccgacgaacaactcgagatgatggccgcgttcgccgaccaggccgcgctggcttggcaattggccacttcgcaacgtcggatgcgcgaactcgacgtactgaccgaccgggatcgtatcgcccgtgacctccatgaccatgtcatccagcggctcttcgcgattggcctggctttgcagggtgctgtcccgcacgaacgtaatcctgaagtgcagcaacgactctcggacgtggtagacgatctgcaagacgttatacaggaaatccggaccaccatttatgacctgcacggagcatcgcagggtatcactcggctccggcagcgaatcgatgcggccgtagcccaatttgccgactcggggttgcgcaccagcgttcaattcgtgggtccattgtcggtggtcgacagcgcgctcgccgatcaggccgaggcggtggttcgggaagcggtcagcaacgcggttcgccatgcgaaggccagcacgttgaccgtccgggtcaaagtcgacgacgacttgtgcatcgaggtgaccgacaacggccgcgggctgcccgacgagttcaccggaagcggcttaacgaacctgcggcagcgggcagagcaggccggcggcgaattcaccctcgcgagcgtaccgggcgcgagcggaacagtgctgcgatggtcagcaccgttgtcgcag

SEQ ID NO: 8 - protein, 578 aa, Mycobacterium tuberculosis
MTTGGLVDENDGAAMRPLRHTLSQLRLHELLVEVQDRVEQIVEGRDRLDGLVEAMLVVTAGLDLEATLRAIVHSATSLVDARYGAMEVHDRQHRVLHFVYEGIDEETVRRIGHLPKGLGVIGLLIEDPKPLRLDDVSAHPASIGFPPYHPPMRTFLGVPVRVRDESFGTLYLTDKTNGQPFSDDDEVLVQALAAAAGIAVANARLYQQAKARQSWIEATRDIATELLSGTEPATVFRLVAAEALKLTAADAALVAVPVDEDMPAADVGELLVIETVGSAVASIVGRTIPVAGAVLREVFVNGIPRRVDRVDLEGLDELADAGPALLLPLRARGTVAGVVVVLSQGGPGAFTDEQLEMMAAFADQAALAWQLATSQRRMRELDVLTDRDRIARDLHDHVIQRLFAIGLALQGAVPHERNPEVQQRLSDVVDDLQDVIQEIRTTIYDLHGASQGITRLRQRIDAAVAQFADSGLRTSVQFVGPLSVVDSALADQAEAVVREAVSNAVRHAKASTLTVRVKVDDDLCIEVTDNGRGLPDEFTGSGLTNLRQRAEQAGGEFTLASVPGASGTVLRWSAPLSQ

SEQ ID NO: 9 - protein, 141 aa, Streptomyces coelicolor
MTRRIRDVMSPAAVAVEPMTTVARAARLMREEDVGDVLVTYDCDLFGVLTDRDIVLRGVADGRDSEATTVGAVCTPPPVVTLEPDDTTDRAAELMARHAVRRLPVVEHGGVPVGVVTLGDLAATDDPHSALADISRAAPGH

SEQ ID NO: 10 - protein, 138 aa, Pseudomonas aeruginosa
MKISDIMTRNVQVADPQQSIREAAATMARIDSGALLVGEGDRLVGMITDRDIAIRAVAGGLSGDTPLGRIMSGDIHYCFEDEDVQHVARNMADIQMRRLPVLNREKRLVGVVSLGNIASCRDQASSATVLQGVAQAHY

SEQ ID NO: 11 - protein, 140 aa, Bacillus subtilis
MSSVKDTMTTQVATVSPNQTIQEAASLMKQHNVGAIPVVEQGVLKGMLTDRDIALRTTAQGRDGQTPVSEVMSTELVSGNPNMSLEDASQLMAQHQIRRLPIVDQNNLVGIVALGDLAVNQMSNESAGSALTNISHQNIH

SEQ ID NO: 12 - DNA, 21 bp, Artificial Sequence
  Primer
gccaccacccttcccgttcag

SEQ ID NO: 13 - DNA, 24 bp, Artificial Sequence
  Primer
atgtcgtcctcgtcagcacctacc

SEQ ID NO: 14 - DNA, 20 bp, Artificial Sequence
  Primer
tcgtaggtgtaggcgggttc

SEQ ID NO: 15 - DNA, 19 bp, Artificial Sequence
  Primer
cggcgatctgcttgttggt

SEQ ID NO: 16 - DNA, 19 bp, Artificial Sequence
  Primer
ggcagccgttcccacattg

SEQ ID NO: 17 - DNA, 20 bp, Artificial Sequence
  Primer
ggctgatcgcgcaccaccac

SEQ ID NO: 18 - DNA, 19 bp, Artificial Sequence
  Primer
ccaccgcacgcgacatcat

SEQ ID NO: 19 - DNA, 18 bp, Artificial Sequence
  Primer
cggaacacggcggacctg

SEQ ID NO: 20 - DNA, 20 bp, Artificial Sequence
  Primer
gcctgggaaactgggtctaa

SEQ ID NO: 21 - DNA, 20 bp, Artificial Sequence
  Primer
tctccacctaccgtcaatcc